(19) United States
(12) Patent Application Publication
(10) Pub. No.: US 2025/0222467 A1
(43) Pub. Date: Jul. 10, 2025

(54) Collection unit for an air filtration system, air filtration system and method of producing a collection element

(71) Applicant: Teknologian tutkimuskeskus VTT Oy, Espoo (FI)

(72) Inventors: Ilpo Kulmala, Espoo (FI); Kimmo Heinonen, Espoo (FI); Hannu Salmela, Espoo (FI)

(21) Appl. No.: 18/848,936

(22) PCT Filed: Mar. 20, 2023
(86) PCT No.: PCT/FI2023/050158

§ 371 Date: Sep. 20, 2024

(30) Foreign Application Priority Data
Mar. 21, 2022 (FI) 20225249

(51) Int. Cl. B03C 3/47; B03C 3/60; B05D 1/04

(52) U.S. Cl. CPC B03C 3/47; B03C 3/60; B05D 1/04

(57) ABSTRACT

A collection unit, an air filtration system, and a method of producing a collection element for a collection unit is herein proposed for adsorbing hazardous or undesired gases as well as collecting airborne particles. The novel collection unit for an air filtration system includes a plurality of collection elements that are configured to provide surfaces to attract airborne particles by electric charge, wherein said surfaces of said collection elements are treated with a chemisorption compound. In the novel method, a collection element is provided and a gas flow is provided over the collection element surface. A chemisorption compound, such as one based contained in a liquid solution, is aerosolised to form an aerosol and, subsequently, the aerosol formed in the gas flow is deposited on the surfaces of said collection element.

### FIELD

The present invention relates to an air filtration system and a method of removing airborne particles and gaseous impurities by means of physisorption and chemisorption.

### BACKGROUND

WO 2010026018 A1 discloses a method for removing pollutants from an exhaust gas stream of an internal combustion engine that uses a fuel containing sulfur, wherein the exhaust gas stream contains at least 1,000 mg of sulfur oxides (SOx) per standard cubic meter and wherein the method comprises the steps of: contacting the exhaust gas stream with a solid adsorbent or with a scrubbing solution, wherein acidic pollutants, including sulfur dioxide and sulfur trioxide, are bound chemisorptively; adding at least a portion of a reducing agent or a chemical precursor thereof to the exhaust gas stream; then passing the exhaust gas stream through a particle filter, wherein organic and inorganic particles are separated; and then feeding the exhaust gas stream to a catalyst, wherein nitrogen oxides are catalytically converted by way of the reducing agent added to the exhaust gas. The device according to the invention comprises a desulfurization stage, a particle filtration stage and a denitrification stage.

US 2018015482 A1 discloses an electronic air filter containing two separate parts: an inlet part and an outlet part; an ionizer located in the inlet part; a collector located in the outlet part; the inlet part is detachable from the outlet part. The inlet part is a door. The outlet part contains detachable set of collecting electrodes.

GB 895683 A discloses a process for deodorizing air comprises the odour-containing air in contact with a water adsorbing activated material, e.g. silica gel alumina, silica-alumina, activated bauxite, and activated clays having a permanganate, e.g. of potassium, sodium, magnesium, calcium, barium and lithium deposited in the pores thereof

Odour-filled air is drawn from rooms by ducts and inlet and passes to a filter bed disposed in the latter and comprising a porous container filled with loosely packed adsorbent material which has been treated with permanganate, where the odours are absorbed by the bed and oxidized by the permanganate. A heat-exchanger assembly, for example located in the basement of the house and adapted to air condition the rooms comprises a heat-exchange element which may be a heating or cooling element, and a blower for passing air over the heat-exchange element into an outlet for distribution to the rooms by means of ducts respectively. Treatment of exhaust gases for internal combustion engines and deodorizing of spaces in which foods are stored may also be effected using the same compositions and methods.

The current existing air filters have the capability of removing particulate contaminants from an air stream; however, they lack the capability to remove gaseous pollutants simultaneously when filtering the air current. There are existing chemical filters which rely on using activated carbon to adsorb the gaseous pollutants but they suffer a drawback of high material cost and significant pressure drop in filtration which leads to high energy consumption, making them unappealing for use in heating, ventilation and air conditioning systems.

### SUMMARY

The invention is defined by the features of the independent claims. Some specific embodiments are defined in the dependent claims.

According to a first aspect of an embodiment, there is provided a collection unit for an air filtration system, the collection unit comprising a plurality of collection elements that are configured to provide surfaces to attract airborne particles by electric charge, wherein said surfaces of said collection elements have been treated with a chemisorption compound.

The collection elements can be made of electrically conductive or static dissipative material. One of the functions of the collection elements is its physical adsorption and/or physisorption ability. Physical adsorption is the result of a relatively weak interaction between the collection element surface and the gas through attractive forces. The attractive forces involve relatively weak van der Waals forces and low heats of adsorption usually not exceeding 80 kJ/mole. The advantage of physical adsorption is that it does not affect the structure or texture of the adsorbent therefore the longevity of the collection unit is good for its application. In addition, desorption can take place when conditions are reversed so the collected particles can be washed away after collection.

The collection elements may be further provided with gas adsorptive properties with the use of chemisorption compounds. The chemisorption compound is being deposited on the surfaces of said collection elements. One further result of deposition of the chemisorption compound is that said compound is being impregnated in said collection elements.

The deposited chemisorption compound can be in the form of dendrites or stalagmites. The dendrites and stalagmites structures for the deposited chemisorption compound would offer optimal amount of quantity, strength and heterogeneity of chemisorption sites and those in turn determine the rate of chemisorption process of a collection element. The higher the number, strength or heterogeneity of chemisorption sites the higher the rate of chemisorption process.

The chemisorption compound can be an oxidant and/or a catalyst. The chemisorption compound can be any one or a combination of sulphuric acid, phosphoric acid, potassium carbonate, iron oxide, potassium, iodide, tri-ethylene di-amine, sulphur, potassium permanganate, manganese IV oxide, silver, zinc oxide, chromium silver salts, copper silver salts, mercury II chloride, Zinc acetate, and noble metals; the examples are for exemplary purposes and are not intended to be an exhaustive list.

When the chemisorption compound is used as an oxidant it would allow partial oxidation to take place at the collection elements' surfaces. Manganese, cobalt, bismuth, iron, copper, and silver are catalysts that are effective in gas-phase oxidation. Gas-phase oxidation of ammonia, methane, ethylene, and propylene would then be made possible.

Chemisorption compounds essentially provide chemisorption functionality to the collection element. The benefit of chemisorption is that the collection elements can be made selective regarding the types of chemicals that it can adsorb or absorb. The chemisorption sites tend to be a single layer process that is localized at the collection element. The chemisorption sites, which can be referred as reactive sites, may be homogeneous or heterogeneous according to the energy characteristics of the adsorption site. The rate of chemisorption process can be improved at elevated temperatures.

In the case if a catalyst is used for the chemisorption then the catalyst can accelerate a chemisorption reaction without being consumed in the process. A homogeneous catalyst is one which is of the same phase as the reactant; for example, a liquid-liquid solution. Alternatively, heterogeneous catalyst is one which is of different phase as the reactant; for example, a solid-gas interaction.

Each collection element can be connected to an electric potential in order to form an active electric field between the collection elements. The collection elements are arranged in parallel manner to each other such that the collection elements are oriented to extend in a direction of flow of gas to be filtered. The electric field would provide the necessary magnetic forces to attract the charged particles towards the collection elements thereby adsorbing them onto their surfaces.

According to another embodiment, there is provided a method of producing a collection element for a collection unit having treated with a chemisorption compound. In the method it comprises providing a collection element, and over the collection element surface is provided with a gas flow. A chemisorption compound, such as one based contained in a liquid solution, is aerosolised to form an aerosol and, subsequently, the aerosol formed in the gas flow is deposited on the surfaces of said collection element.

The chemisorption compound in the form of aerosol is then deposited by electrically charging said collection element. The generated aerosol can also be electrically charged. The charged aerosol in combination with the collection element being charged make it possible to deposit aerosol on targeted locations of surfaces of said collection element to be controlled by varying electric field strength of the collection element. The aerosol constitutes mainly of non-volatile residuals of the chemisorption compound. The aerosolizing of the solution can be carried out by means of a spray, a coating gun, or an ultrasonic nebulizer.

According to another embodiment, a method of producing a collection unit for a filtration unit whereby in a housing for channelling air flow is provided with a plurality of collection elements that are pre-treated with chemisorption chemicals, and a corona charging unit. The method described hereinbefore can be used to pre-treat the collection elements so that each collection element can be produced as a single unit. The collection elements can then be modified to a particular specification adapted for a particular application.

According to one embodiment the chemisorption compound is deposited on the surfaces of said collection elements.

According to one embodiment each collection element is connected to an electric potential such that an electric field is formed between the collection elements.

At least some of the described embodiments overcome the shortcomings of the present existing air filtration system by offering a combination of physisorption and chemisorption properties in a collection element. The result of a new method of producing a collection element having physisorption and chemisorption properties is a more efficient and effective air filtration system with the capability to adsorb micro-particles suspended in air as well as removing odorous gaseous particles or substances at low energy consumption.

### BRIEF DESCRIPTION OF THE DRAWINGS

FIGS. 1A and 1B illustrate a sectional view of a collection unit with the collection elements having surfaces to attract airborne particles by electric charge in accordance with at least some embodiments.

FIG. 2 illustrates one configuration of the collection elements being arranged adjacent to each other in a parallel manner according to one embodiment.

### DETAILED DESCRIPTION OF EMBODIMENTS

In the present context, the term “electrically conductive” comprises objects or type of material that allows the flow of an electrical current in one or more directions. The materials can be metallic such as copper or any other types of metals belong in the periodic table or, non-metallic such as graphite or polymers that are conductive known as conductive polymers.

In the present context, the term “electric charge” comprises positive and negative charges. The electric charge can be exhibited by electrostatic attraction or repulsion in the presence of other matter. The attraction or repulsion forces are the resultant effect a matter experiences in response to its exposure to an electromagnetic field.

In the present context, the term “aerosolise” comprises vaporising, atomising, subliming and other way of converting a substance from a liquid suspension to gaseous form.

In the present context, the term “corona discharge” comprises a process by which a current flows from an electrode with a high potential into a neutral fluid by ionizing that fluid so as to create a corona charging zone around the electrode. The ions generated eventually pass charge to nearby areas of lower potential, or recombine to form neutral gas molecules.

In the present context, the term “static dissipative” means that the charges flow to ground more slowly and in a somewhat more controlled manner than with conductive materials. Resistivity generally between 10⁶ and 10⁹ ohms per square. Low or no initial charges prevent discharge from human contact. The material may be either surface-coated or filled throughout.

In the present context, the term “electrostatic discharge” (ESD) comprises the sudden flow of electricity between two electrically charged objects caused by contact, an electrical short, or dielectric breakdown. A build-up of static electricity can be achieved by tribo-charging or by electrostatic induction.

In the present context, the term “plasma” is an artificially electromagnetic field where a gaseous substance becomes increasingly electrically conductive once passing through the field.

In the present context, the term “dendrite” means a crystal or crystalline aggregates that possess multi-branching form which resembles a tree-like structure. Dendritic crystallization forms a natural fractal pattern. One example of dendritic crystal growth is snowflake formation and frost patterns.

In the present context, the term “stalagmite” means polycrystalline aggregates that are arranged in successive, well-stacked layers.

In the present context, the term “chemisorption” means a chemical reaction that allows a strong chemical interaction between the adsorbed molecule and the surface molecule of the active site.

In the present context, the term “physisorption” means the molecules are attracted by van der Waals forces, and attach themselves to the surface of the solid.

In one embodiment, there is provided a collection unit 120 for an air filtration system 100, the collection unit 120 comprising a plurality of collection elements 122, 123 that are configured to provide surfaces to attract airborne particles 210 by electric charge, wherein said surfaces of said collection elements 122, 123 are treated with a chemisorption compound.

Air surrounding our environment is typically polluted with airborne particles 210. These airborne particles 210 are made up of micro particles such as dust particles or other fine particles that can be of any origin from inorganic matters to organic matters. The airborne particles are often neutral in charges when suspended in air but they can be charged electrostatically. These airborne particles can become charged 211, 212 by passing through corona charging zone 113, 114 exhibited by a corona charging unit 110 as these particles flow through an air filter system. The corona charging zone 113, 114 is an ion flow region, a part of which contains or may contain plasma. The corona charging zone 113, 114 may be created by opposing electrodes (not illustrated) that charge the volume of the corona charging zone 113, 114. This charging of the particles allows them to be subsequently attracted towards a surface. It is possible to substitute the corona charging unit 110 with other types of ionisers for ionising particles in any one embodiment.

The collection elements 122, 123 provide a surface area to which the airborne particles 210 are attracted. The collection elements 122, 123 act as an adsorbent thus having adsorptive properties. The airborne particles 210 are being attracted to the collection elements' 122, 123 surfaces through electrostatic differences between the two entities. This process is described as physical adsorption or referred as physisorption, where the atoms, ions or molecules from a gas, liquid or dissolved solid, are being adhered to a surface of the collection elements 122, 123.

The collection elements 122, 123 can be made of insulative, electrically conductive or static dissipative material. The materials are chosen according to their ability to protect and/or prevent ElectroStatic Discharge (ESD).

Insulative materials have the benefit of preventing or limiting the flow of electrons across their surface or through their volume. Insulative materials have a high electrical resistance and are difficult to ground. Static charges remain in place on these materials for a relatively longer duration than conductive materials. Insulative materials are defined as those having a surface resistivity of at least 1×10¹² Ω/sq or a volume resistivity of at least 1×10¹¹ Ωcm.

According to one embodiment, one way of providing the chemisorption compound to the collection elements is by depositing on the surfaces of said collection elements 123,124. In addition to or as an alternative method of depositing on the surfaces, the chemisorption compound can be impregnated in the said collection elements 122, 123 by wetting for example. Specific examples of wetting include dipping and spraying. In the case where the chemisorption compounds are being provided to the collection elements then the collection elements 122, 123 can act both as an adsorbent and as an absorbent simultaneously. Gaseous pollutants in the airstream can diffuse onto the treated surfaces and they are subsequently reacted chemically with the chemisorption compounds impregnated in the collection elements thus being trapped by chemisorption. At the same time, the airborne micro-particles 210 can be collected onto the collection elements through physisorption.

The deposited chemisorption compound can be deposited on the surface of the collection elements in the form of dendrites or stalagmites using the method described hereinbefore. The chemisorption compound in the form of stalagmite typically compose of crystals ≥1 mm wide and ≥2 mm long, elongated perpendicular to the growing speleothem surface, with a length to width ratio of about 6:1, and it can have serrated or open crystal boundaries. The inter-crystalline of stalgamite possess pores, of which the pores can be uniform or non-uniform in size. The stalgamite comprises also regular stacking of rhombohedra crystallites. The benefit of the stalagamite as a form of crystalline structure for the chemisorption compound is that with its porous structure offers a large amount of reactive sites for chemisorption.

The chemisorption compounds used are oxidant and/or catalyst. The chemisorption compound can be selected from any one or more of the following: sulphuric acid, phosphoric acid, potassium carbonate, iron oxide, potassium, iodide, tri-ethylene di-amine, sulphur, potassium permanganate, manganese IV oxide, silver, zinc oxide, chromium silver salts, copper silver salts, mercury II chloride, Zinc acetate, and noble metals; the examples are for exemplary purposes and are not intended to be an exhaustive list.

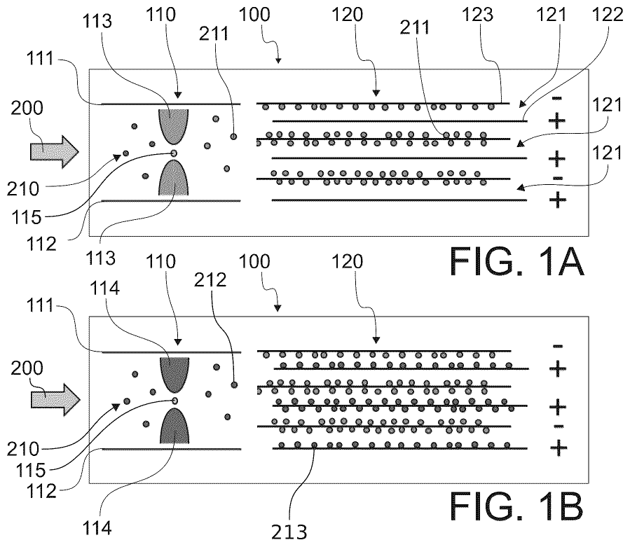

Each collection element 122, 123 can be connected to an electric potential through the use of electrodes in order to provide an active electric field between the collection elements 122, 123. In the examples of FIGS. 1A and 1, the collection elements 122 have a positive potential and the collection elements 123 have a negative potential for attracting negatively charged particles 212 and positively charged particles 211, respectively. The collection elements 122, 123 are arranged in parallel manner to each other such that the collection elements 122, 123 are oriented to extend in a direction of flow of gas 200 to be filtered. It is not necessary that the collection elements to be absolutely adjacent to each other such that the collection element can be partially overlapped. In other words, one collection element can be overlapped with more than one other collection element.

An air filtration system 100 enclosed inside a housing comprises a corona charging unit 110 and a collection unit 120. The housing is configured to channel an air flow 200. The collection unit 120 comprises or is made up of a series of collection elements 122, 123 having been treated with chemisorption compounds. The housing is essentially the main body of air filtration system 100 comprising an airflow conduit having an airflow inlet for connection to an air supply duct, and an airflow outlet. Mounting means can be provided to the housing in order to allow the main body to be attached to a structure of a building in an easily accessible location. The corona charging unit 110 and a collection unit 120 can be mounted directly to the main body such that the air filtration system can be installed as a single unit.

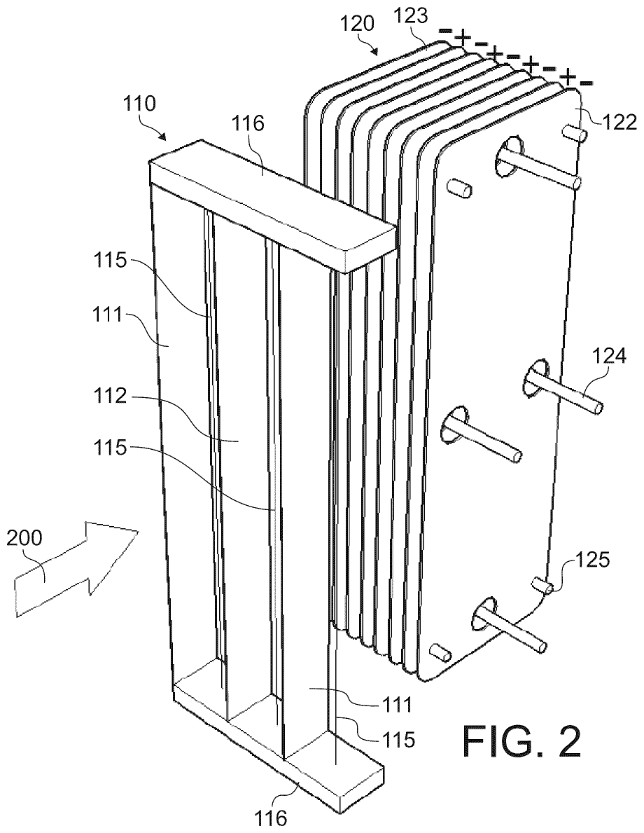

FIG. 2 illustrates one configuration of the collection elements 122, 123 being arranged adjacent to each other in a parallel manner according to one embodiment. FIG. 2 also shows the collection elements 122, 123 with high-voltage electrodes 124 and grounding electrodes 125 running through respective openings on the collection elements 122, 123. The grounding electrodes 125 carry the collection elements 122, 123 between two grounding plates (not shown in the drawings).

FIG. 2 also illustrates an exemplary construction of the charging unit 110. The charging unit 110 features alternating ioniser surfaces 111, 112 and corona wires 115 extending within a frame 116. The ioniser surfaces 111, 112 and intermediate corona wires 115 are spaced apart from each other to create a sufficient air gap for receiving the incoming air flow 200 carrying airborne particles. Such charging units 110 are known per se.

In another embodiment, the collection elements 122, 123 can be of any planar geometry such as plates and other plate-like forms. The geometry of the collection elements are so that the air flow 200 is not interrupted to allow a laminar flow. This configuration should be chosen in such a way that it would prevent a pressure drop in the filtration process.

One configuration is that the collection elements 122, 123 are arranged in a parallel manner such that it involves a gap 121 between the collection elements in a direction perpendicular to the planar surface of the collection elements 122, 123. In one parallel configuration, the plates are in a spiral form. The most effective configuration of a collection unit would comprise a large number of parallel collection elements. And the collection elements are arranged in such a way that they are in close separation between each other to provide an air gap 121 with maximal contact surface with the air flow 200. The air gap 121 depends on the collecting electric field strength and on the pressure drop of the filter. A foreseeable air gap 121 may be in the range of 1 to 5 mm.

One alternative arrangement of the collection elements 122, 123 is that they are provided in the form of corrugated material in a rolled up configuration to form a cylindrical body. The cylindrical body can be made up of at least two corrugated materials. The corrugated form offers yet further extension of surface area for the collection elements.

Another embodiment of the collection elements 122, 123 are that they are in a rolled up configuration to form a cylindrical body where the cylindrical body can be made up of at least two rolled up planar collection elements with air gaps 121 in between and the corrugated materials are provided in the air gaps. The corrugated materials, in this case, provide structural support for the separations of surface area of the collection elements.

Alternatively, the collection elements separated by the corrugated material is not rolled up to a spiral configuration, but produced by additive manufacturing. With the availability of manufacturing methods including 3D printing, the collection elements 122, 123 can adopt other forms with high surface area such as single-centred cylinders between which the insulation, such as air, or monocular polygons or honeycomb. In other words, the collection elements 122, 133 may co-centric profiles that have a circular or polygon cross-section. There can be a corrugated or otherwise meandering support structure between the collection elements 122, 123. The collection elements 122, 123 can adopt also other forms and configurations as long as those configurations provide channels for an air flow 200 which do not disrupt an air flow of the air to be filtered.

FIGS. 1A and 1B illustrate, in accordance with at least one embodiment, the aerosol particles can be fed in a gas flow 200 through corona charging zone 113, 114, exhibited by a corona charger, so that they can become either positively or negatively charged 211, 212. FIG. 1A shows the corona charging zone 113 being positively charged, whereas FIG. 1B shows the corona charging zone 114 being negatively charged. The polarity between positive and negative may be swapped at least once, i.e. one or more times during the charging process. The strength of the electric field may also be changed according to a case-specific profile so as to achieve uniform deposition. The charged particles with their corresponding charges are then being attracted to either collection element with their respective opposite charges.

According to another embodiment, the method of treating a collection element 122, 123 uses a gas flow. In the gas flow is contained a liquid solution of chemisorption compound that has been aerosolized by means of a spray, a coating gun, or an ultrasonic nebulizer. The aerosol constitutes mainly of non-volatile residuals of the chemisorption compound.

The liquid solution of chemisorption compound can be prepared before being aerosolised. The liquid solution is a suspension of chemisorption compound in a solvent. Water may be used as the solvent as it does not react chemically with the compounds and because it is easy to apply. The solvent to be used is so that it is immiscible to the chemisorption compound in order to allow the suspension to be formed. The solvent also need to be volatile in order to be evaporated once the liquid solution has been aerosolised.

During the particle injection phase, the liquid solution containing the dissolved chemisorption compounds is sprayed. When the solvent evaporates from the sprayed droplets, the remained residuals which constitute mainly the non-volatile chemisorption compound are being fed through an electrostatic precipitator. The residuals are then being charged in the charging section and then subsequently being deposited on the collection elements.

According to an alternative embodiment, the chemisorption compound is in the form of powder which is aerosolized and the deposited onto the collection elements 122, 123.

The chemisorption compound in the form of aerosol is deposited by electrically charging said collection element 122, 123. The charged aerosol can be deposited on targeted areas of the charged collection element's surface 122, 123 by varying electric field strength of the collection element 122, 123. The electric field can be amplified to higher magnitudes until it has the desired strength to draw in the charged aerosol electrostatically onto the collection element, resulting in an even distribution of chemisorption compound being deposited on the collection elements.

The electric field strength of the collection element can have an effect on the quality of deposition of the chemisorption compounds onto the collection element. By varying the electric field strength of the collection element, different densities of deposition of the chemisorption compounds can be achieved.

In another embodiment, the chemisorption compound is impregnated into the collection element 122, 123. The impregnation of the chemisorption is ideally carried out to a point of saturation. The degree of saturation varies depending on the material that the collection element is made of, e.g., plastic, metal, cardboard, activated carbon or zeolite. The material of the collection elements is preferably insulative, electrically conductive or static dissipative.

### EXAMPLES

10% citric acid solvent is used to dissolve a chemisorption compound to make up a solution to be sprayed. A spray gun has a maximum spray rate of 100 ml/min is used to spray the solution. The solution in the form of liquid aerosol is then led into an air filter system with an air flow of 10-20 L/s. The target air stream has a temperature of 50-60 C. The non-volatile chemisorption compound in the liquid aerosol is then charged by passing through corona charging zone of an ioniser. The wire charger has field strength of 8 kV/˜1 mA. The filter supply voltage is +100-8000 V. Filter leakage current during spraying is 0-70 μA.

The following table provides three different filters with using different conditions of preparation in the deposition of chemisorption compounds.

| | | | | Leakage |
|---|---|---|---|---|
| | | Face | | current |
| | Air flow | velocity | Filter supply | @2 kV |
| Sample | (l/s) | (m/s) | voltage (kV) | (μA) |
| Filter 1 | 19.7 | | ±(0.1, 0.3, | ±(18, 20, |
| | | | 0.5, 1) | 36, 50) |
| Filter 2 | 10 | | ±(0.1, 0.3, | <100 |
| | | | 0.5, 1) | |
| Filter 3 | 10 | 1.1 . . . 1.7 | ±(1, 2, 4, 8) | <100 |

The following table illustrates the filtration efficiency test result for the same three filters using 1 mol citric acid (192 g) reacted with 3 mol ammonia (51 g).

| | | | | Expected |
|---|---|---|---|---|
| | Initial | | Pressure | life cycle |
| | efficiency | Duration | difference | (ambient conc. |
| Filter | (%) | (min) | (Pa) | 20 ppb) |
| 1 | 68 | 76 | 75 | ~100 h |
| 2 | 77 | >25 | 16 | |
| 3 | 50 | 80 | 22 | |

The following table provides the examples of chemisorption compounds that can be used for the deposition onto the collection elements and their applications.

| Impregnation | | | |
|---|---|---|---|
| | Quan- | | |
| | tity | Activated | |
| Chemicals | (wt %) | Carbon³ | Examples for application |
| Sulfuric acid | 2-26 | F 1-4 mm Ø | Ammonia, amine, mercury |
| Phosphoric acid | 10-30 | F 1-4 mm Ø | Ammonia, amine |
| Potassium | 10-20 | F 1-4 mm Ø | Acid gases (HCl, HF, |
| carbonate | | | SO₂, H₂S, NO₂), |
| | | | carbon disulfide |
| Iron oxide | 10 | F 1-4 mm Ø | H₂S, mencaption COS |
| Potassium | 1-5 | F 1-4 mm Ø | H₂S, PH₃, Hg, |
| iodide | | | AsH₃, radioactive |
| | | | gases/radioactivemethyl |
| | | | iodide |
| Triethylene | 2-5 | F 1-2 mm Ø | Radioactive gases/ |
| diamine | | G 6-16 mesh | radioactivemethyl |
| (TEDA) | | | iodide |
| Sulfur | 10-20 | F 1-4 mm Ø, G | mercury |
| Potassium | 5 | F 3 + 4 mm Ø | H₂S from oxygen- |
| permanganate | | | lacking gases |
| Manganese | | G 6-16 mesh | Aldehyde |
| IV oxide | | | |
| Silver | 0.1-3 | P 3 + 4 mm Ø | P: phosphine, arsine |
| | | G 6-30 mesh | G: domestic drinking |
| | | | water filters |
| | | | (oligodynamic effect) |
| Zinc oxide | 10 | F 1-4 mm Ø | Hydrogen cyanide |
| Chromium- | 10-20 | F 0.6-3 mm Ø | Civil and military |
| copper- | | G 12-30 mesh | gas protection |
| silver salts | | G 6-16 mesh | Phosphine, chlorine, |
| | | | arsine |
| | | | Chloropierin, sarin |
| | | | and other nerve gases |
| Mercury II | 10-15 | F 3 + 4 mm Ø | Vinyl chloride synthesis |
| chloride | | | Vinyl fluoride synthesis |
| Zinc acetate | 15-25 | F 3 + 4 mm Ø | Vinyl acetate synthesis |
| Noble metals | 3-5 | F, G, P | Organic synthesis |
| (palladium, | | | hydrogenation |
| platinum) | | | |

F = pelletized activated carbon
G = granulated activated carbon
P = powdered activated carbon
Ø = pellet diameter

The collection elements can be pre-treated with chemisorption chemicals using the method described hereinbefore. The treated collection elements have a long shelf life, in terms of years, and they can be stored under normal condition without suffering any degeneration in chemisorption and physisorption characteristics. The treated collection elements can be independently installed in other air filters, but it would require modifications to accommodate them in order to have the right configurations to maximise its functionality.

An air particle filter/collection unit produced using the method described hereinbefore would provide additional capability to remove targeted gaseous pollutants from the filtered air. One potential application area of the filter is in recirculating air filters where high clean air delivery rates and thus significant improvements in the indoor air quality can be achieved even at modest single-pass removal efficiencies.

Removal of both particulate and gaseous pollutants from the building supply air means that it is possible to significantly reduce the entry of outdoor contaminants indoors. Healthier indoor air quality is increasingly more appreciated in polluted urban areas as the public awareness of the adverse health effects due to poor air quality is increasing. The filtration can be realised at low pressure drops meaning low energy consumption. Thanks to these characteristics the indoor airborne pollutants can be collected more efficiently and cost-effectively than ever before.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### INDUSTRIAL APPLICABILITY

An air particle filter/collection unit produced using the method in the present invention to provide additional capability to remove targeted gaseous pollutants from the filtered air. One potential application area of the filter is in recirculating air filters with high clean air delivery rates. The present invention offers significant improvements in the indoor air quality even at modest single-pass removal efficiencies.

### REFERENCE SIGNS LIST

| No. | Feature |
|---|---|
| 100 | air filtration system |
| 110 | ioniser |
| 111 | ioniser surface |
| 112 | ioniser surface |
| 113 | positively charged corona charging zone |
| 114 | negatively charged corona charging zone |
| 115 | corona wire |
| 116 | frame |
| 120 | collection unit |
| 121 | air gap |
| 122 | positively charged collection element |
| 123 | negatively charged collection element |
| 124 | high-voltage electrode |
| 125 | grounding electrode |
| 200 | air flow |
| 210 | airborne particle |
| 211 | positively charged particle |
| 212 | negatively charged particle |
| 213 | adsorbed negatively charged particle |

### CITATION LIST

### Patent Literature

US 2018015482 A1
GB 895683 A
WO 2010026018
U.S. Pat. No. 7,998,259 B2
U.S. Pat. No. 7,160,363 B2

### Non Patent Literature

Paper: K. Sim, et al., “Antimicrobial nanoparticle coated electrostatic air filter with high filtration efficiency and low pressure drop”, Science of the Total Environment 533 (2015) 266-274. This study provides valuable information for the development of a hybrid air purification system that can serve various functions and be used in an indoor environment.

1. A collection unit for an air filtration system, the collection unit comprising a plurality of collection elements that are arranged in parallel manner to each other and configured to provide surfaces to attract airborne particles by electric charge, wherein said surfaces of said collection elements are treated with a chemisorption compound.

2. The collection unit according to claim 1, wherein the collection elements:
are made of insulative, electrically conductive or static dissipative material or
have gas adsorptive properties, or
are made of insulative, electrically conductive or static dissipative material and have gas adsorptive properties.

3. The collection unit according to claim 1, wherein the chemisorption compound is deposited on the surfaces of said collection elements, preferably in the form of dendrites or stalagmites.

4. The collection unit according to claim 1, wherein the chemisorption compound is impregnated in the said collection elements.

5. The collection unit according to claim 1, wherein the chemisorption compound is an oxidant and/or a catalyst.

6. The collection unit according to claim 5, wherein the chemisorption compound is any one or more selected from the group comprising:
citric acid C₆H₈O₇,
potassium hydroxide KOH,
sodium hydroxide NaOH,
sodium carbonate Na₂CO₃,
sodium bicarbonate NaHCO₃,
potassium bicarbonate KHCO₃,
sodium thiosulfate Na₂S₂O₃,
hopcalite, mixture of MnO₂ and CuO,
sulphuric acid,
phosphoric acid,
potassium carbonate,
iron oxide,
potassium,
iodide,
triethylene diamine,
sulphur,
potassium permanganate,
manganese IV oxide,
silver,
zinc oxide,
chromium silver salts,
copper silver salts,
mercury II chloride,
Zinc acetate, and
noble metals.

7. The collection unit according to claim 1, wherein the collection elements are oriented to extend in a direction of flow of gas to be filtered.

8. The collection unit according to claim 7, wherein: the collection elements are plates and, wherein the parallel arrangement involves a gap between the plates in a direction perpendicular to the planar surface of the collection elements,
the collection elements are in the form of corrugated material arranged in a rolled up configuration to form a cylindrical body, or
the collection elements are co-centric profiles, e.g. with a circular or polygon cross-section.

9. The collection unit according to claim 8, wherein the cylindrical body is made up of at least two corrugated materials.

10. An air filtration system comprising:
a corona charging unit, and
a collection unit provided downstream of the corona charging unit, which collection unit comprises a plurality of collection elements that are arranged in parallel to each other and configured to provide surfaces to attract airborne particles by electric charge, wherein said surfaces of said collection elements are treated with a chemisorption compound.

11. The air filtration system according to claim 10, wherein the air filtration system comprises a housing that encloses the corona charging unit and a collection unit.

12. A method of producing a collection element for a collection unit, the method comprising:
providing a collection element with parallel collection element surfaces,
providing a gas flow over the collection element surfaces,
aerosolizing a chemisorption compound to form an aerosol in the said gas flow, and;
depositing the chemisorption compound suspended in said aerosol from the gas flow on the surfaces of said collection element.

13. The method according to claim 12, wherein in the aerosolization step, a liquid solution of chemisorption compound is aerosolized to form the aerosol.

14. The method according to claim 12, wherein said aerosol is deposited by electrically charging said collection element for producing an electric field there between.

15. The method according to claim 12, wherein the generated aerosol is electrically charged.

16. The method according to claim 12, wherein the depositing of aerosol on targeted locations of surfaces of said collection element is carried out by varying electric field strength between adjacent collection elements.

17. The method according to claim 15, wherein said aerosol constitutes mainly of non-volatile residuals of the chemisorption compound.

18. The method according to claim 15, wherein the aerosolizing of the solution is carried out by means of a spray, a coating gun, or an ultrasonic nebulizer.
